(19) United States
(12) Patent Application Publication
(10) Pub. No.: US 2026/0096868 A1
(43) Pub. Date: Apr. 9, 2026

(54) Apparatus for Mounting a Fiducial Marker for Image Guided Procedure and Method for Mounting the Same

(71) Applicant: Daniel H. Kim, Houston, TX (US)

(72) Inventor: Daniel H. Kim, Houston, TX (US)

(21) Appl. No.: 19/349,813

(22) Filed: Oct. 3, 2025

Related U.S. Application Data
(60) Provisional application No. 63/703,918, filed on Oct. 5, 2024.

(51) Int. Cl. A61B 90/00

(52) U.S. Cl. CPC A61B 90/39; A61B 2090/3987; A61B 2090/3991

(57) ABSTRACT

An apparatus for mounting a fiducial marker for an image guided procedure according to the present disclosure includes a fiducial marker unit fixed to an in-body target, a guide unit formed of a flexible member extending longitudinally and having one end connected to the fiducial marker unit, and a holder unit detachably connected to the fiducial marker unit through the guide unit to fix the fiducial marker unit to the in-body target or remove the fiducial marker unit from the in-body target.

### TECHNICAL FIELD

The present disclosure relates to an apparatus for mounting a fiducial marker for an image guided procedure and a method for mounting the same, and more particularly, to an apparatus for mounting a fiducial marker capable of rapidly and safely fixing the fiducial marker to an in-body target and removing the fiducial marker from the in-body target without obstructing the field of view during the placement of the fiducial marker during upon a surgery or procedure including image guided procedures, and a method for mounting the same.

### BACKGROUND ART

In various medical conditions involving structural or functional damage to tissues in the body, surgery or procedures may be necessary to restore or remove damaged organs or bone tissue.

The above-described surgeries or procedures have evolved to include image guided procedures (IGPs) utilizing imaging modalities such as endoscopes, ultrasound, X-rays, CT, and MRI and improved instruments, with the goal of achieving minimal invasiveness by minimizing incisions of skin and soft tissue.

For example, image guided surgery (surgical navigation system, hereinafter IGS) for spinal treatment may include an image guided procedure that registers preoperatively acquired tomographic imaging data (e.g., CT, CBCT, or MRI) with a patient's actual position during surgery and tracks spatial coordinates of a surgical instrument in real time through a localization system to accurately map spatial coordinates to anatomical structures within the image.

Furthermore, image guided procedures (interventional radiology, hereinafter IR) for local tumor treatment, such as drainage tube insertion, embolization, vascular intervention such as stenting, radiofrequency ablation (RFA), and cryotherapy, may also include an image guided procedure that uses real-time images, such as ultrasound, fluoroscopy, and CT, as a guide to precisely guide surgical instruments, such as needles or catheters, to target sites.

As a result, a user can precisely guide surgical instruments to a target site while viewing a navigation screen.

In this case, in both the IGS and IR, the fiducial marker serves as a reference point to enhance registration and tracking accuracy.

Specifically, as illustrated in FIG. 1, in the IGS, bone-anchored fiducial markers are installed on specific anatomical targets T, such as spinous process, posterior arch, and ilium, before or during surgery, and utilized for the image guided procedure.

In addition, in the IR, various types of fiducial markers M, such as seed, coil, or clip types, may be inserted into target lesions or adjacent soft tissues.

The above-described fiducial marker M is inserted in various ways depending on a surgical site and an application area.

For example, the bone-anchored fiducial markers M, which is used for precise registration of a spine and skeletal system, is typically inserted by drilling a small hole after a primary incision, followed by final fixation of a screw-or pin-shaped marker to the bone tissue using a dedicated mounting mechanism.

When the fiducial marker M remains in the body after the surgery or procedure is completed, complications such as local discomfort, foreign body reactions, increased risk of infection, imaging artifacts, and interference with additional procedures (e.g., additional surgeries or procedures, MRI, etc), or persistent discomfort, may occur.

When it is determined that reconfirmation of the position of the fiducial marker M at the completion of the surgery or procedure is difficult due to bleeding, swelling, or limitations of the visual field, or that the risk outweighs the benefit because of the burden of additional incision or dissection required for removal, the above-described fiducial marker M may be left in place in its entirety.

In addition, partial separation or residual fiducial markers may occur due to soft tissue burrowing, bone overgrowth, or the like.

To alleviate the above-described problems, a form in which a rigid post P is extended from the fiducial marker M is employed to enhance intraoperative identification, grasping, and accessibility for removal. However, the above-described method may obstruct a user's field of vision and a surgical instrument flow.

In addition, when the post P is subjected to external pressure, it may cause microfractures or soft tissue damage due to a lever effect, and may also induce unnecessary scattering or obstruction (LoS) on the navigation screen N, thereby potentially leading to reduced accuracy of the surgery or procedure.

Therefore, a method for solving these problems is required.

### DISCLOSURE

### Technical Problem

The present disclosure has been devised to solve the above-described problems of the prior art, and an object of the present disclosure is to provide an apparatus for mounting a fiducial marker capable of rapidly and safely fixing the fiducial marker to an in-body target and removing the fiducial marker from the in-body target without obstructing the field of view during the placement of the fiducial marker during upon a surgery or procedure including image guided procedures, and a method for mounting the same.

Problems of the present disclosure are not limited to the above-described problems. That is, other problems that are not described may be obviously understood by those skilled in the art from the following specification.

### Technical Solution

To achieve the above-described above, according to the present disclosure, an apparatus for mounting a fiducial marker for an image guided procedure may include: a fiducial marker unit fixed to an in-body target; a guide unit formed of a flexible member extending longitudinally and having one end connected to the fiducial marker unit; and a holder unit detachably connected to the fiducial marker unit through the guide unit to fix the fiducial marker unit to the in-body target or remove the fiducial marker unit from the in-body target.

Here, the holder unit may be formed in a hollow tube shape so that the guide unit is inserted thereinto and moves, engaged with the fiducial marker unit through the guide unit inserted thereinto to fix the fiducial marker unit to the in-body target, and separated from the fiducial marker unit and removed from the body to expose a portion of the guide unit outside the body.

In this case, the holder unit may insert the guide unit, which is exposed outside the body, into the body, and then may be engaged with the fiducial marker unit along the inserted guide unit to remove the fiducial marker unit from the body.

Meanwhile, according to the present disclosure including the above-described configuration, the guide unit may be formed as the flexible member extending longitudinally, and thus may be maintained in a non-rigid state when the fiducial marker unit and the holder unit are separated.

Here, according to the present disclosure, the apparatus for mounting a fiducial marker for an image guided procedure may further include a sheath portion provided to have greater rigidity than the holder unit and enclosing the holder unit.

Here, the sheath portion may be provided to enclose at least a section of the holder unit along a longitudinal direction thereof.

In addition, the sheath portion may be provided to maintain a shape deformed by an external force.

Meanwhile, according to the present disclosure including the above-described configuration, the holder unit may be engaged with the fiducial marker unit by any one of mechanical engagements caused by shape interference, friction, elastic deformation, press-fitting, catching, or magnetism.

In this case, the holder unit may be engaged with the fiducial marker unit in an interference fit manner.

In addition, the holder unit may be engaged with the fiducial marker unit in a threaded fastening manner.

Further, the holder unit may be rotationally engaged with the fiducial marker unit in a bayonet coupling.

To achieve the above-described above, according to the present disclosure, a method for mounting a fiducial marker for an image guided procedure is a method for mounting a fiducial marker using the above-described apparatus for mounting a fiducial marker for an image guided procedure. The method may include: engaging the holder unit to the fiducial marker unit connected to one side of the guide unit through the guide unit; fixing the fiducial marker unit to an in-body target through the holder unit; separating the holder unit from the fiducial marker unit; and removing the holder unit from the body so that a portion of the guide unit is exposed outside the body.

In addition, to achieve the above-described above, according to the present disclosure, the method may include: inserting the guide unit exposed outside the body into the holder unit; re-engaging the holder unit to the fiducial marker unit along the inserted guide unit; and retrieving the fiducial marker unit from the body.

### Advantageous Effects

To solve the above-described problems, according to an apparatus for mounting a fiducial marker for an image-guided procedure and a method for mounting the same of the present disclosure, it is possible to rapidly and safely fix the fiducial marker to an in-body target and remove the fiducial marker from the in-body target without obstructing the field of view during the placement of the fiducial marker during upon a surgery or procedure including image guided procedures.

The effects of the present disclosure are not limited to the above-described effects. That is, other effects that are not described may be obviously understood by those skilled in the art from the claims.

### BRIEF DESCRIPTION OF THE DRAWINGS

The summary described above, as well as the detailed description of the preferred embodiments of the present application described below, will be better understood when read in conjunction with the accompanying diagrams.

For the purpose of illustrating the present disclosure, preferred embodiments are illustrated in the diagrams.

However, it should be understood that the present application is not limited to the precise arrangements and means illustrated.

FIG. 1 is a diagram for describing a problem in a conventional image guided procedure.

FIG. 2 is a diagram for describing an apparatus for mounting a fiducial marker for an image guided procedure according to an embodiment of the present disclosure.

FIG. 3 is a diagram for describing a guide unit in the apparatus for mounting a fiducial marker for an image guided procedure according to an embodiment of the present disclosure.

FIG. 4 is a diagram for describing a state in which a fiducial marker is fixed to an in-body target through a holder unit in the apparatus for mounting a fiducial marker for an image guided procedure according to an embodiment of the present disclosure.

FIG. 5 is a diagram for describing a state in which the fiducial marker is fixed to the in-body target and then the holder unit is removed from the body in the apparatus for mounting a fiducial marker for an image guided procedure according to an embodiment of the present disclosure.

FIGS. 6 and 7 are diagrams illustrating a state in which a portion of the guide unit is exposed outside the body in a non-rigid state by separating the holder unit from a fiducial marker unit and removing the holder unit from the body in the apparatus for mounting a fiducial marker for an image guided procedure according to an embodiment of the present disclosure.

FIG. 8 is a diagram for describing a state in which the holder unit is inserted into the body and re-engaged with the fiducial marker unit fixed to the in-body target in the apparatus for mounting a fiducial marker for an image guided procedure according to an embodiment of the present disclosure.

FIG. 9 is a diagram for describing a state in which the fiducial marker unit is removed from the body through the holder unit in the apparatus for mounting a fiducial marker for an image guided procedure according to an embodiment of the present disclosure.

FIG. 10 is a diagram for describing a tape for temporarily fixing the holder unit in the apparatus for mounting a fiducial marker for an image guided procedure according to an embodiment of the present disclosure.

FIG. 11 is a diagram for describing an apparatus for mounting a fiducial marker for an image guided procedure according to another embodiment of the present disclosure.

FIG. 12 is a diagram illustrating a sheath portion in the apparatus for mounting a fiducial marker for an image guided procedure according to another embodiment of the present disclosure.

FIG. 13 is a diagram for describing a method for mounting a fiducial marker for an image guided procedure according to an embodiment of the present disclosure.

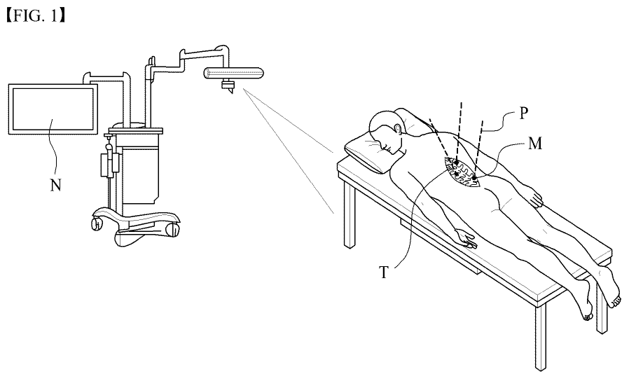

### BEST MODE

Hereinafter, embodiments of the present disclosure will be described in detail with reference to the accompanying diagrams so that those skilled in the art may easily practice the present disclosure. However, description of the present disclosure is only an embodiment for structural or functional description, and therefore the scope of the present disclosure should not be construed as limited to embodiments described in the text. That is, since the embodiments may be variously modified and may have various forms, the scope of the present disclosure should be construed as including equivalents capable of realizing the technical idea. In addition, a specific embodiment is not construed as including all the objects or effects presented in the present disclosure or only the effects, and therefore the scope of the present disclosure should not be understood as being limited thereto.

The meaning of the terms described in the present application should be understood as follows.

Terms such as “first” and “second” are intended to distinguish one component from another component, and the scope of the present disclosure should not be limited by these terms. For example, a first component may be named a second component and the second component may also be similarly named the first component. It is to be understood that when one element is referred to as being “connected to” another element, it may be connected directly to or coupled directly to another element or be connected to another element, having the other element intervening therebetween. On the other hand, it is to be understood that when one element is referred to as being “connected directly to” another element, it may be connected to or coupled to another element without the other element intervening therebetween. Meanwhile, other expressions describing a relationship between components, that is, “between”, “directly between”, “neighboring to”, “directly neighboring to”and the like, should be similarly interpreted.

It should be understood that the singular expression include the plural expression unless the context clearly indicates otherwise, and it will be further understood that the terms “comprises” or “have” used in this specification, specify the presence of stated features, steps, operations, components, parts, or a combination thereof, but do not preclude the presence or addition of one or more other features, numerals, steps, operations, components, parts, or a combination thereof.

Unless defined otherwise, all the terms used herein including technical and scientific terms have the same meaning as meanings generally understood by those skilled in the art to which the present disclosure pertains. It should be understood that the terms defined by the dictionary are identical with the meanings within the context of the related art, and they should not be ideally or excessively formally defined unless the context clearly dictates otherwise.

Hereinafter, exemplary embodiments of the present disclosure in which objects of the present disclosure may be specifically implemented will be described with reference to the accompanying diagrams.

In exemplary embodiments of the present disclosure, the same terms and reference numerals will be used to describe the same components. Therefore, an additional description for the same component will be omitted below.

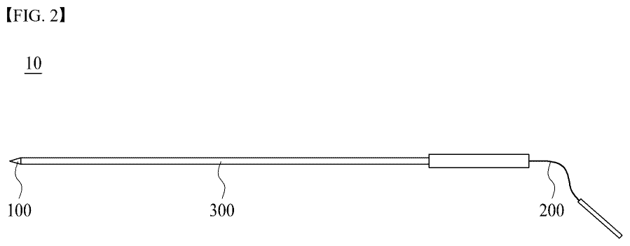

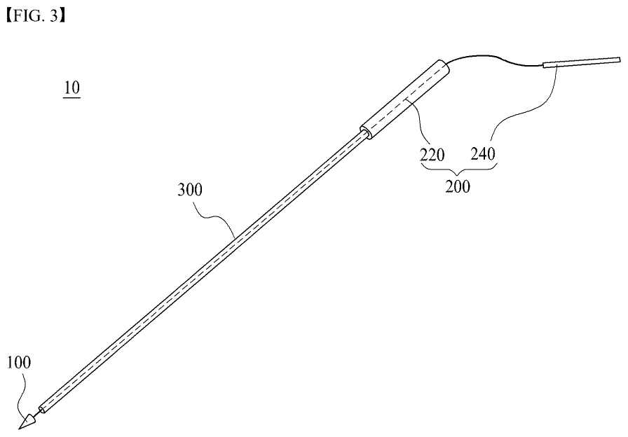

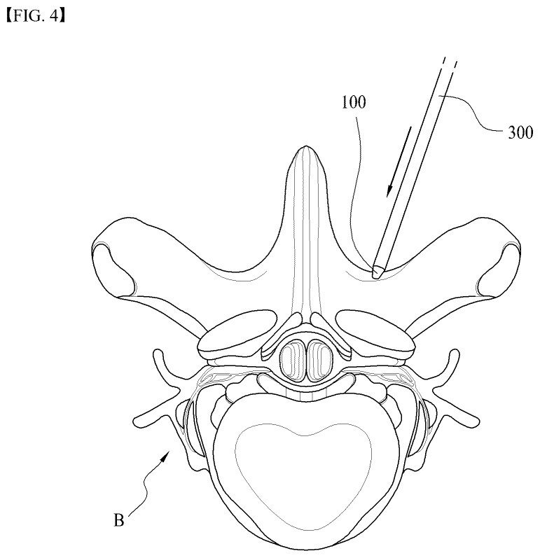

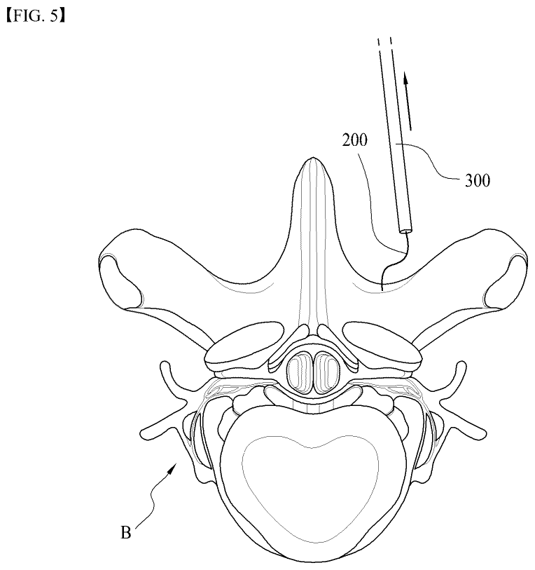

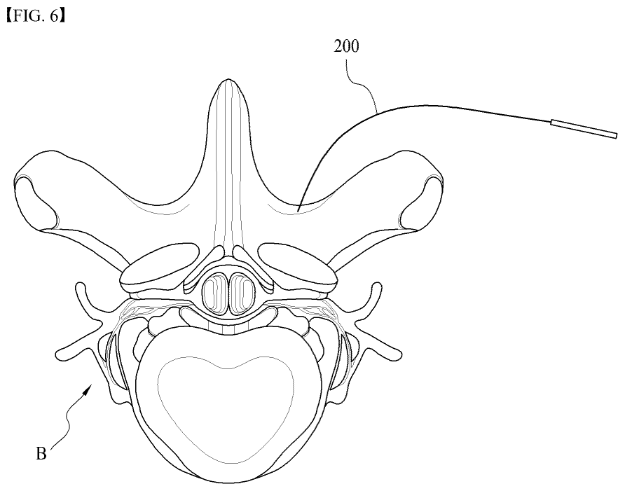

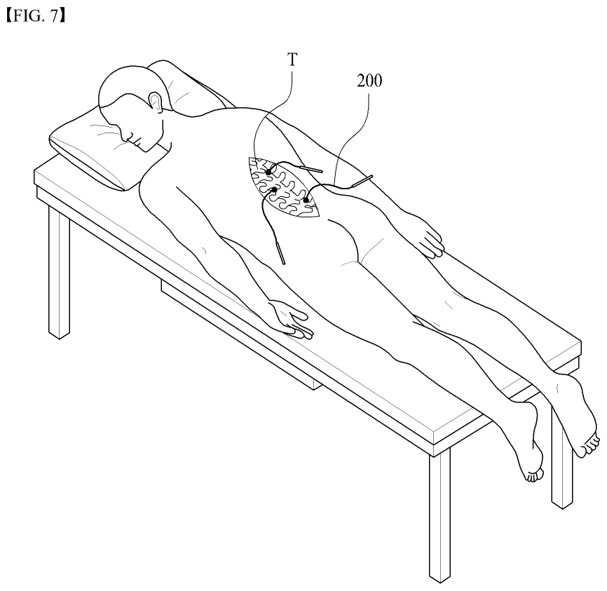

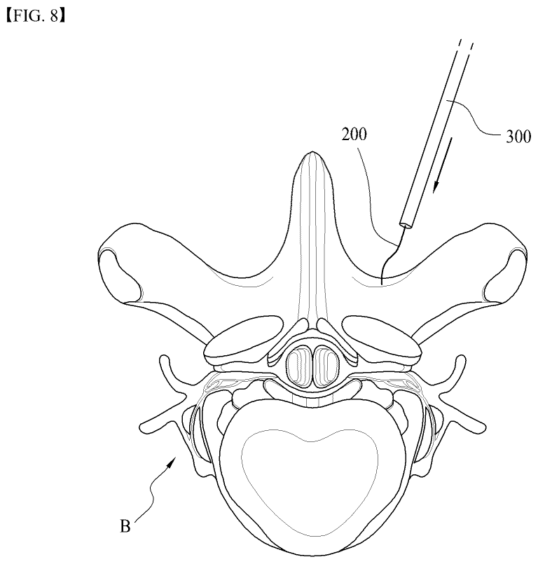

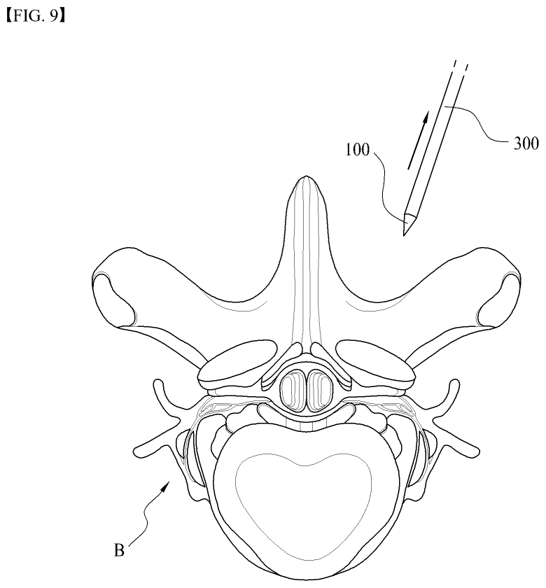

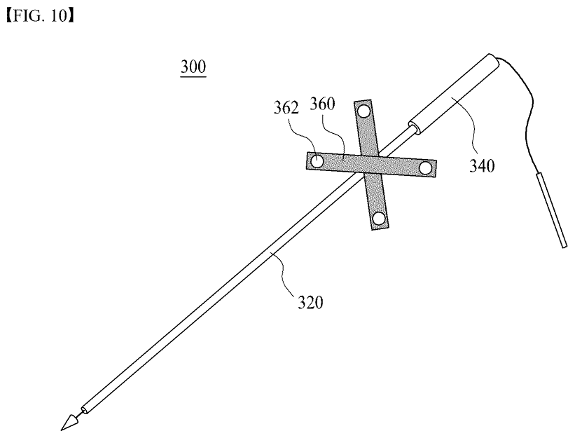

FIG. 2 is a diagram for describing an apparatus for mounting a fiducial marker for an image guided procedure according to an embodiment of the present disclosure, FIG. 3 is a diagram for describing a guide unit in the apparatus for mounting a fiducial marker for an image guided procedure according to an embodiment of the present disclosure, FIG. 4 is a diagram for describing a state in which a fiducial marker is fixed to an in-body target through a holder unit in the apparatus for mounting a fiducial marker for an image guided procedure according to an embodiment of the present disclosure, FIG. 5 is a diagram for describing a state in which the fiducial marker is fixed to the in-body target and then the holder unit is removed from the body in the apparatus for mounting a fiducial marker for an image guided procedure according to an embodiment of the present disclosure, FIGS. 6 and 7 are diagrams illustrating a state in which a portion of the guide unit is exposed outside the body in a non-rigid state by separating the holder unit from a fiducial marker unit and removing the holder unit from the body in the apparatus for mounting a fiducial marker for an image guided procedure according to an embodiment of the present disclosure, FIG. 8 is a diagram for describing a state in which the holder unit is inserted into the body and re-engaged with the fiducial marker unit fixed to the in-body target in the apparatus for mounting a fiducial marker for an image guided procedure according to an embodiment of the present disclosure, FIG. 9 is a diagram for describing a state in which the fiducial marker unit is removed from the body through the holder unit in the apparatus for mounting a fiducial marker for an image guided procedure according to an embodiment of the present disclosure, and FIG. 10 is a diagram for describing a tape for temporarily fixing the holder unit in the apparatus for mounting a fiducial marker for an image guided procedure according to an embodiment of the present disclosure.

First, as illustrated in FIG. 2, an apparatus 10 for mounting a fiducial marker for an image guided procedure according to an embodiment of the present disclosure largely includes a fiducial marker unit 100, a guide unit 200, and a holder unit 300.

The fiducial marker unit 100 may be fixed to an in-body target B during an image guided procedure, including surgery or other procedures, and may serve to stably mark and track a location of the in-body target B.

That is, the fiducial marker unit 100 may be temporarily or semi-permanently installed within the body for the image guided procedure, and may function as a reference point that enables accurate visualization of the in-body target B through linkage with a medical imaging device.

The above-described in-body target B should be precisely located during the surgery or procedure through the image guided procedure.

Accordingly, the fiducial marker unit 100 is fixed to the above-described in-body target B, enabling an operator to fix a field of view and track a position.

Here, the in-body target B may refer to a specific structure within the human body to which the fiducial marker unit 100 is fixed in the present disclosure.

In other words, the in-body target B is a key element in determining the position of the fiducial marker unit 100 and refers to a clinical point where the fiducial marker unit 100 should be fixed in the image guided procedure.

That is, the in-body target B may be hard tissue, specifically the vertebrae.

Furthermore, the in-body target B may be various bone structures such as the skull, femur, or pelvis, and in some cases, may be soft tissue.

The method for fixing the fiducial marker unit 100 to the above-described in-body target B may be implemented in various ways.

For example, the fiducial marker unit 100 may be mechanically fixed to the in-body target B by forming a protrusion, anchor, or hook shape on an outer peripheral surface of the fiducial market unit 100.

Furthermore, the fiducial marker unit 100 may be fixed by forming a screw thread that is engaged with the in-body target B.

As described above, the method for fixing the fiducial marker unit 100 may be modified in various ways and is not limited to the fixing structure or means presented in the specific embodiment.

The fiducial marker unit 100 described above may be manufactured from a biocompatible material to ensure safety even when remaining in the body for long periods of time.

For example, the fiducial marker unit 100 may be made of stainless steel with excellent corrosion resistance and mechanical strength. Alternatively, titanium with excellent lightness and biocompatibility or a cobalt-chromium (Co—Cr) alloy, which is a high-strength alloy, may also be used.

In addition, the fiducial marker unit 100 may be made of a material with superelastic properties, such as a nickel-titanium (Ni—Ti, nitinol) alloy. If necessary, a biocompatible coating may be added to a surface to minimize chemical reactivity with tissue.

Furthermore, the fiducial marker unit 100 is not limited to the metal materials described above, and may be manufactured in combination with biodegradable polymer materials such as polylactic acid (PLA), polyglycolic acid (PGA), and polylactic-co-glycolic acid (PLGA), or non-biodegradable polymer materials such as polyurethane and polyethylene terephthalate (PET).

The biodegradable metal and polymer composite structure described above may be advantageous in simultaneously ensuring mechanical stability and biocompatibility.

In this case, the fiducial marker unit 100 may also include a sensor module.

The sensor module described above may transmit electromagnetic signals to enable position synchronization with an external device, and may also use optical guidance using optical signals or transmit acoustic signals, such as ultrasound, thereby detecting the position within the body from outside in real time.

Next, the guide unit 200 may be formed of a flexible member 220 extending longitudinally, and one side of the guide unit 200 may be connected to the fiducial marker unit 100.

As illustrated in FIG. 3, the fiducial marker unit 100 may include a flexible member 220 extending longitudinally, such as a wire or rod, and one side of the flexible member 220 may be connected to the fiducial marker unit 100.

Furthermore, the fiducial marker unit 100 may include a grip member 240 connected to the other side of the flexible member 220 described above.

Accordingly, a user may easily confirm the position of the fiducial marker unit 100 connected to the guide unit 200 by holding the grip member 240 described above and confirming the position of the guide unit 200 described above.

Furthermore, the user may easily insert the guide unit 200 into the holder unit 300 to be described below through the grip member 240 described above.

Next, the holder unit 300 may be detachably connected to the fiducial marker unit 100 through the guide unit 200.

Accordingly, the user may hold the holder unit 300 to fix the fiducial marker unit 100 to the in-body target B or remove the fiducial market unit 100 from the in-body target B.

Specifically, the holder unit 300 may include a tube 320 and a handle 340.

For example, the holder unit 300 may include a hollow tube 320 so that the guide unit 200 described above may be inserted thereinto and move.

In addition, the handle 340 is connected to one side of the tube 320, so that the user may hold the handle 340 to manipulate the holder unit 300.

Furthermore, the holder unit 300 may be connected to the fiducial marker unit 100 through the guide unit 200 inserted into the holder unit 300, thereby fixing the fiducial marker unit 100 to the in-body target B.

Furthermore, the holder unit 300 may be separated from the fiducial marker unit 100 and removed from the body, thereby exposing a portion of the guide unit 200 outside the body.

Next, the holder unit 300 inserts the exposed guide unit 200 into the body, and then is connected to the fiducial marker unit 100 along the inserted guide unit 200, thereby removing the fiducial marker unit 100 from the body.

For example, as illustrated in FIG. 4, the user may first insert the other end of the guide unit 200 into the holder unit 300.

Next, by pulling the other end of the guide unit 200 inserted into the holder unit 300 from the outside, the fiducial marker unit 100 may be positioned on one side of the holder unit 300.

That is, the holder unit 300 may be engaged with the fiducial marker unit 100 while moving along the guide unit 200.

Consequently, the guide unit 200 may serve to guide the holder unit 300 to the fiducial marker unit 100.

Next, the user may manipulate the holder unit 300 to insert the holder unit 300 into the body, and fix the fiducial marker unit 100 connected to the holder unit 300 to the in-body target B.

As described above, after the fiducial marker unit 100 is stably fixed to the in-body target B, as illustrated in FIG. 5, the holder unit 300 may be separated from the fiducial marker unit 100 and removed from the body.

That is, the guide unit 200 described above is formed of the flexible member 220 extending longitudinally, so that when the fiducial marker unit 100 and the holder unit 300 are separated, the guide unit may be maintained in a non-rigid state as illustrated in FIGS. 6 and 7.

Therefore, during the surgery or procedure, a portion of the guide unit 200 connected to the fiducial marker unit 100 and exposed to the body may be maintained in the non-rigid state, thereby ensuring a clear field of vision and preventing interference with instrument movement, and minimizing interference from external forces.

The term “non-rigid” as described herein may include a state in which, even when protruding outside the body, the unit does not remain upright rigidly but may easily sag or bend under the influence of gravity or an external force.

For example, this non-rigid state may be achieved when the guide unit 200 is processed into a shape such as a thread, wire, string, or hollow filament with a smaller diameter than the holder unit 300.

Furthermore, the above-described non-rigid state may include a state in which the guide unit 200 is limp and flexible such that, even without any force applied by the user, the guide unit 200 may not stand by itself and may be displaced or bent on its own.

Furthermore, the above-described non-rigid state includes the state in which, even if the guide unit 200 is deformed into an arbitrary shape by an external force and maintains that shape, the guide unit 200 may not maintain an upright position on its own.

For example, when the guide unit 200 is processed into the shape of a bendable wire, an articulable rod, or a malleable strand that maintains its shape after bending, this may also correspond to a non-rigid state.

The holder unit 300 described above may be formed of a flexible or rigid material, and may be appropriately selected depending on the surgical or treatment environment.

However, the holder unit 300 should be stable and easily manipulated by the user during the process of fixing or removing the fiducial marker unit 100 within the body.

In this case, when the holder unit 300 is too flexible, force may not be transmitted in the desired direction during the insertion or manipulation within the body, or it may be difficult to accurately fix the in-body target B.

Therefore, to ensure ease and reliability of manipulation within the body, it is convenient to manufacture the holder unit 300 using a rigid material.

As illustrated in FIGS. 8 and 9, when removing the fiducial marker unit 100 from the in-body target B, the guide unit 200 exposed outside the body is inserted back into the holder unit 300, and then the holder unit 300 moves along the inserted guide unit 200 toward the fiducial marker unit 100, and the holder unit 300 and the fiducial marker unit 100 are engaged, so the fiducial marker unit 100 fixed to the in-body target B may be removed from the body.

Since the holder unit 300 described above is removed after fixing the fiducial marker unit 100 to the in-body target B, the interference during the surgical procedure is minimized.

However, the guide unit 200 exposed outside the body is highly flexible, and therefore, depending on the type or location of the in-body target B, the guide unit 200 may stimulate the surrounding affected area, potentially causing an infection risk or unnecessary damage.

Considering the above-described possibility, as illustrated in FIG. 10, the holder unit 300 may include the tube 320, the handle 340, and a tape 360.

The tape 360 described above may help minimize the above-described possibility by temporarily attaching the exposed guide unit 200 to the skin and stabilizing the guide unit 200.

In this case, the tape 360 may further include an additional marker 362.

Therefore, the position of the holder unit 300 may be confirmed during surgery through the additional marker 362 provided on the tape 360.

As a result, even when the holder unit 300 is temporarily attached to the skin, the accuracy of the image guided procedure may be improved.

The tape 360 described above may be applied to human skin in various materials, shapes, etc., and the scope of the present disclosure is not limited thereto.

In other words, the tape 360 described above is not limited in material or shape as long as it may be applied to human skin, and the scope of the present disclosure is not limited thereto.

However, for a more specific description, as an example, the tape 360 may be formed in an X shape and made of a biocompatible material such as polyurethane, polyethylene, or non-woven fabric, which may be advantageous in terms of fixation strength, skin adhesion, breathability, and ease of use.

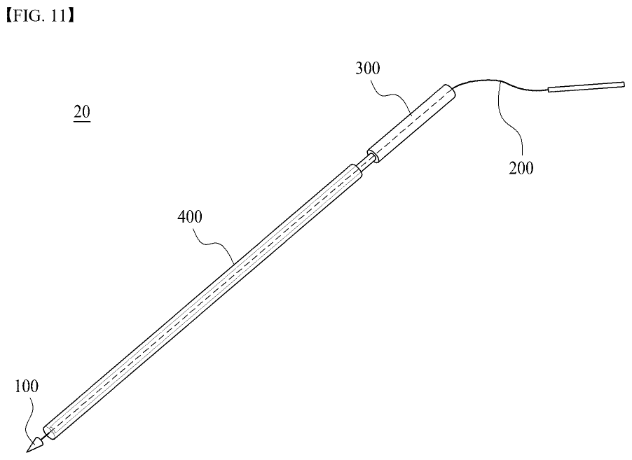

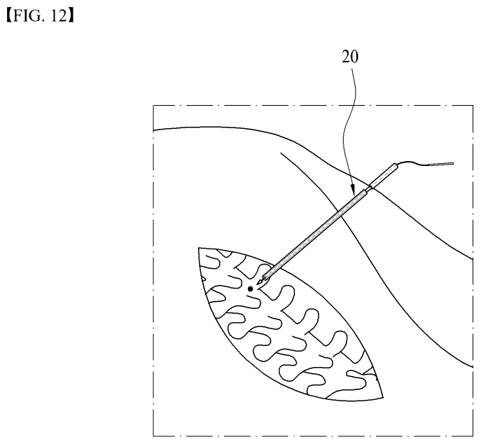

Meanwhile, FIG. 11 is a drawing illustrating an apparatus 20 for mounting a fiducial marker for the image guided procedure according to another embodiment of the present disclosure, and FIG. 12 is a drawing illustrating a sheath portion 400 in the apparatus 20 for mounting a fiducial marker for an image guided procedure according to another embodiment of the present disclosure.

As illustrated in FIG. 11, the apparatus 20 for mounting a fiducial marker for the image guided procedure according to another embodiment of the present disclosure largely includes the fiducial marker unit 100, the guide unit 200, and the holder unit 300, and may further include a sheath portion 400.

Here, the fiducial marker unit 100, the guide unit 200, and the holder unit 300 are identical to the fiducial marker unit 100, the guide unit 200, and the holder unit 300 described in an embodiment of the present disclosure, and thus, a detailed description thereof will be omitted.

Hereinafter, the sheath portion 400, which is different from the first embodiment, will be described in detail.

The sheath portion 400 is formed to have relatively greater rigidity than the holder unit 300 and is structured to enclose the holder unit 300 from the outside.

Therefore, excessive deformation or shaking that may occur when the holder unit 300 is used alone may be effectively suppressed.

For example, the sheath portion 400 may be formed to be disposed at least a section along the longitudinal direction of the holder unit 300.

That is, the sheath portion 400 may be configured to enclose the entire holder unit 300, including the handle 340, or, as illustrated in FIG. 12, may be configured to enclose only the tube 320 of the holder unit 300.

Also, although not illustrated in the drawing, the flexibility and rigidity of the holder unit 300 may be simultaneously controlled by disposing the sheath portion 400 only in a localized portion of the tube 320 of the holder unit 300, if necessary.

The above-described partial reinforcement structure may enhance usability and provide efficiency in the manufacturing and assembly process.

When the holder unit 300 described above is formed of a flexible material, the sheath portion 400 described above may be formed of a flexible material, but may be formed of a material with relatively high rigidity compared to the holder unit 300.

In this case, the sheath portion 400 is formed of a polyether block amide (Pebax) material, which simultaneously provides appropriate flexibility and excellent mechanical properties, thereby preventing the bending of the holder unit 300.

Furthermore, the sheath portion 400 is formed of a polytetrafluoroethylene (PTFE) material, which has a low coefficient of friction and excellent chemical resistance, thereby reducing frictional resistance that may occur during the insertion into the body and maintaining the stable performance even over long periods of use.

Furthermore, the sheath portion 400 may be configured as a multilayer structure including the Pebax material described above and the PTFE material described above.

At least one of the above-described sheath portions 400 may be selectively disposed along the longitudinal direction of the holder unit 300.

Therefore, the sheath portion 400 may suppress unnecessary bending of the holder unit 300 and simultaneously improve precision during the insertion and fixation of the fiducial marker.

Furthermore, the sheath portion 400 may be provided to maintain a shape deformed by an external force.

Meanwhile, in the apparatuses 10 and 20 for mounting a fiducial marker for an image guided procedure according to the above-described embodiments of the present disclosure, the holder unit 300 may be engaged with the fiducial marker unit 100 by any one of shape interference, friction, elastic deformation, press-fitting, catching, or magnetic mechanical engagement.

As used herein, “physical engagement” refers to a state in which two members are connected to each other by a physical force or action.

The physical bonding described above may include various methods, including mechanical engagement, chemical bonding, and electrical engagement at the target site.

The mechanical engagement refers to a form in which two members are engaged and connected through interactions such as shape, friction, elastic deformation, press-fit, catching, and magnetism.

For example, the screw fastening, snap-fit, press-fit, interference fit, frictional engagement, etc., are all specific embodiments of the mechanical engagement.

The mechanical engagement described above may be implemented without additional components, offering structural simplicity and ensuring safety even when used in the body.

In the present disclosure, a configuration in which the fiducial marker unit 100 and the holder unit 300 are connected through mechanical engagement is described, but this is merely an example.

When the fiducial marker unit 100 and the holder unit 300 may be detachably connected, regardless of whether the engagement is mechanical, chemical, or electrical, such engagement may fall within the scope of the present disclosure.

That is, the fiducial marker unit 100 and the holder unit 300 may be connected through the various engagement methods described above.

However, the chemical or electrical engagement inevitably requires additional components for engagement.

In other words, the chemical or electrical engagement poses a risk of the additional components becoming detached or detached within the body.

The chemical bonding refers to a state in which components are engaged together by chemical action, such as adhesives, adhesive films, welding, or chemical coatings.

The chemical bonding method described above requires additional bonding materials or components, such as adhesive application or welding processes. Therefore, while structurally stable, problems may arise in the body when the adhesive or chemical components become detached or detached.

Furthermore, the electrical engagement refers to a form in which components are connected through conductive materials or electrical action.

Examples thereof may include conductive adhesives, soldering, and connector pin engagement.

The electrical engagement method described above also requires additional engagement members, which may lead to safety issues when these members become detached or detached within the body.

In summary, since the chemical bonding or electrical engagement may pose challenges to patient safety and the reliability of the procedure, in particular, the present disclosure proposes a configuration in which the fiducial marker unit 100 and the holder unit 300 are connected via the mechanical engagement.

The mechanical engagement described above enables stable engagement and separation without the need for additional members, thereby ensuring both the safety and efficiency during the medical procedures.

The in-body environment in which the fiducial marker is fixed is generally very confined and has characteristics that make it difficult to directly observe with the naked eye.

Under these limited conditions, minor operational inconveniences experienced by the user during insertion or removal of the holder unit 300 may be significantly amplified, which may directly impact the accuracy of the procedure and patient safety.

Therefore, the connection structure between the fiducial marker unit 100 and the holder unit 300 is not simply an optional element, but rather may be an essential element that enables rapid and accurate fastening and disassembly while maintaining minimal invasiveness.

In other words, the connection method that may operate reliably even in limited space and field of view conditions should be fixed to simultaneously meet the dual requirements of procedure efficiency and reduced patient burden.

For this purpose, the fiducial marker unit 100 and the holder unit 300 may be engaged using any of the following methods: interference fit, threaded fastening, or bayonet coupling.

Furthermore, when the fiducial marker unit 100 and the holder unit 300 are connected using the interference fit, guide members may protrude from the fiducial marker unit 100 and the holder unit 300 along the insertion direction to guide the interference fit.

Furthermore, when the fiducial marker unit 100 and the holder unit 300 are engaged using a threaded fastening method, A multi-start screw may be formed on the fiducial marker unit 100 and the holder unit 300 so that the fiducial marker unit 100 and the holder unit 300 may be fastened together.

Here, unlike the conventional screw, the multi-start screw has a structure where the threads are not in a single row but exist simultaneously in multiple rows.

For example, a 2-start screw has two rows of threads starting 180° apart, while a 3-start screw has three rows of threads starting 120° apart.

As described above, when multiple threads are arranged in parallel, even screws of the same diameter can advance farther forward with just one full rotation.

Therefore, a user may quickly and easily engage the fiducial marker unit 100 and the holder unit 300, even in confined spaces or environments with poor visibility.

Furthermore, after engagement, since multiple threads simultaneously distribute the force, it is not easily disengaged by external force and can remain securely fixed.

Furthermore, since it may be easily detached with just a short rotation, both the engagement and separation are efficient.

Furthermore, to ensure quick and accurate attachment and detachment while maintaining minimal invasiveness, a bayonet coupling method is adopted, enabling rapid attachment and detachment by rotating only a predetermined angle after insertion.

Meanwhile, when the in-body target B is the hard tissue described above, and the fiducial marker unit 100 may be stably fixed to the in-body target B, the shape of the fiducial marker unit 100 may be designed in various ways depending on the fixation method, as described above.

For example, when the fiducial marker unit 100 has a screw-like structure, the fiducial marker unit 100 may be fixed by screwing into the bone. Conversely, when the fiducial marker unit 100 has a non-screw-like wedge-shaped structure, it may be inserted into the bone and fixed by the friction force or press-fit force.

In particular, when the fiducial marker unit 100 is formed into the wedge-shaped structure, a diameter of the fiducial market unit 100 may be modified to form a non-linear curve along the insertion direction.

The shape of the non-linear curve described above may be, for example, an exponential function, a curve with a gradually changing curvature, or a shape with a sharp decrease in the middle. Due to such a shape, the fiducial marker unit 100 may be easily entered during the insertion, and after fixation, the interference force with the bone increases, thereby providing the stable fixing effect.

Furthermore, when the external force is applied to the holder unit 300, the fiducial marker unit 100 may be firmly fixed to the bone, but relatively smoothly separated when removed. To this end, the fiducial marker unit 100 may be provided with a low-friction coating, or additionally formed with self-tapping protrusions for cutting the bone during insertion to enhance the fixing force.

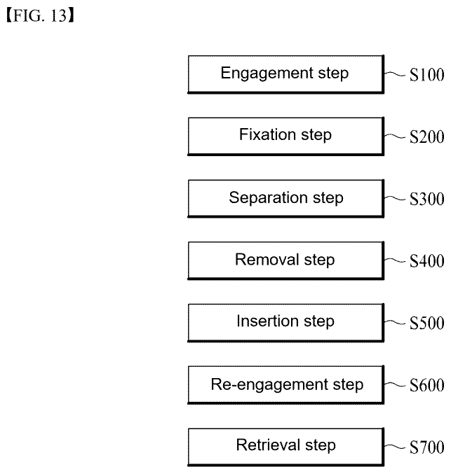

FIG. 13 is a diagram for describing a method for mounting a fiducial marker for an image guided procedure according to an embodiment of the present disclosure.

The configuration and operation process of the above-described apparatuses 10 and 20 for mounting a fiducial marker for an image guided procedure are as described above.

Based on this, the following describes a method for mounting a fiducial marker according to an embodiment of the present disclosure, focusing on the procedural flow.

Referring to FIG. 13, the method of mounting a fiducial marker for the image guided procedure according to an embodiment of the present disclosure largely includes an engagement step (S100), a fixation step (S200), a separation step (S300), and a removal step (S400).

In the engagement step (S100), a user inserts the other end of the guide unit 200 into the holder unit 300. Then, when the guide unit 200 is pulled from outside the body, the fiducial marker unit 100 is positioned on one side of the holder unit 300. That is, the holder unit 300 moves along the guide unit 200 and is engaged with the fiducial marker unit 100. As a result, the guide unit 200 serves to guide the holder unit 300 to the fiducial marker unit 100.

In the fixation step (S200), the user inserts the combined holder unit 300 into the body and then fixes the fiducial marker unit 100 to the in-body target B via the holder unit 300.

In the above-described fixation step (S200), the external force applied to the holder unit 300 is transmitted to a force that pushes the fiducial marker unit 100 to the target location, thereby stably inserting and fixing the fiducial marker unit 100 to the target tissue.

As a result, the fiducial marker unit 100 may mark the target location without shaking during the image guided procedure.

In the separation step (S300), after the fiducial marker unit 100 is stably fixed to the in-body target B, the user may separate the holder unit 300 from the fiducial marker unit 100.

In this case, the guide unit 200 is maintained in the state in which the guide 200 is connected to the fiducial marker unit 100 and partially exposed outside the body. Therefore, even after the holder unit 300 is separated, the fiducial marker unit 100 remains fixed to the target.

In the removal step (S400), the separated holder unit 300 is completely removed from the body. In this case, a portion of the guide unit 200 remains protruding from the body, which may be used as a path for retrieving the fiducial marker unit 100 later.

In particular, since the guide unit 200 is formed of the flexible member 220, the exposed portion is maintained in the non-rigid state, does not obstruct the field of vision during the procedure, and minimizes interference with the movement of other surgical instruments.

In addition to the mounting steps described above, the method of mounting a fiducial marker for an image guided procedure according to an embodiment of the present disclosure may further include an insertion step (S500), a re-engagement step (S600), and a retrieval step (S700), which are performed upon the completion of the surgery or procedure.

That is, after the medical procedure related to the image guided procedure is completed while the fiducial marker unit 100 is fixed to the in-body target B, the guide unit 200, which is exposed outside the body, is inserted into the holder unit 300. Through this, the holder unit 300 may be re-engaged with the fiducial marker unit 100, and then a procedure of retrieving the fiducial marker unit 100 outside the body may be performed.

In the insertion step (S500), the user re-inserts a portion of the guide unit 200, which is exposed outside the body, into the holder unit 300.

That is, the insertion step (S500) is a procedure for preparing the holder unit 300 to be reconnected to the fiducial marker unit 100 along the guide unit 200.

In the re-engagement step (S600), the holder unit 300 may be moved along the inserted guide unit 200 to be re-engaged with the fiducial marker unit 100.

The re-engagement step (S600) corresponds to a preparatory procedure in which, while the fiducial marker unit 100 is already fixed inside the body, the holder unit 300 is re-engaged so that the fiducial marker unit 100 may be withdrawn outside the body.

Finally, in the retrieval step (S700), the fiducial marker unit 100 may be removed from the body by pulling the re-engaged holder unit 300.

In the above-described retrieval step (S700), the fiducial marker unit 100 is safely withdrawn along the guide unit 200 and may be retrieved so that the fiducial marker unit 100 does not remain unnecessarily in the body after the procedure is completed.

The preferred embodiments according to the present disclosure have been reviewed, and the fact that the present disclosure can be embodied in other specific forms without departing from the spirit or scope in addition to the above-described embodiments is obvious to those skilled in the art.

Therefore, the above-described embodiments should be considered as illustrative rather than restrictive, and accordingly, the present disclosure is not limited to the above description but may be modified within the scope of the appended claims and their equivalents.

In other words, embodiments of the present disclosure are not implemented only through the apparatus and/or operating method described above, and may be implemented through a program that realizes functions corresponding to the configuration of the embodiments of the present disclosure or a recording medium in which the program is recorded. Such an implementation can be easily implemented by those skilled in the art to which the present disclosure pertains based on the description of the above-described embodiment. Although embodiments of the present disclosure have been described in detail hereinabove, the scope of the present disclosure is not limited thereto, but may include several modifications and alterations made by those skilled in the art using a basic concept of the present disclosure as defined in the claims.

### DESCRIPTION OF REFERENCE NUMERALS

B: Target site in body
10: Apparatus for mounting a fiducial marker for an image guided procedure
100: Fiducial marker unit
200: Guide unit
220: Flexible member
240: Grip member
300: Holder unit
320: Tube
340: Handle
360: Tape
362: Additional marker
20: Apparatus for mounting a fiducial marker for an image guided procedure
400: Sheath portion
S100: Engagement step
S200: Fixation step
S300: Separation step
S400: Removal step
S500: Insertion step
S600: Re-engagement step
S700: Retrieval step

1. An apparatus for mounting a fiducial marker for an image guided procedure, comprising:
a fiducial marker unit fixed to an in-body target;
a guide unit formed of a flexible member extending longitudinally and having one end connected to the fiducial marker unit; and
a holder unit detachably connected to the fiducial marker unit through the guide unit to fix the fiducial marker unit to the in-body target or remove the fiducial marker unit from the in-body target.

2. The apparatus of claim 1, wherein the holder unit is formed in a hollow tube shape so that the guide unit is inserted thereinto and moves,
is engaged with the fiducial marker unit through the guide unit inserted thereinto to fix the fiducial marker unit to the in-body target, and
is separated from the fiducial marker unit and removed from the body to expose a portion of the guide unit outside the body.

3. The apparatus of claim 2, wherein the holder unit inserts the guide unit, which is exposed outside the body, into the body, and then is engaged with the fiducial marker unit along the inserted guide unit to remove the fiducial marker unit from the body.

4. The apparatus of claim 1, wherein the guide unit is formed as the flexible member extending longitudinally, and thus maintains a non-rigid state when the fiducial marker unit and the holder unit are separated.

5. The apparatus of claim 4, further comprising:
a sheath portion provided to have greater rigidity than the holder unit and enclosing the holder unit.

6. The apparatus of claim 5, wherein the sheath portion is provided to enclose at least a section of the holder unit along a longitudinal direction thereof.

7. The apparatus of claim 5, wherein the sheath portion is provided to maintain a shape deformed by an external force.

8. The apparatus of claim 4 or 5, wherein the holder unit is engaged with the fiducial marker unit by any one of mechanical engagements caused by shape interference, friction, elastic deformation, press-fitting, catching, or magnetism.

9. The apparatus of claim 8, wherein the holder unit is engaged with the fiducial marker unit in an interference fit manner.

10. The apparatus of claim 8, wherein the holder unit is engaged with the fiducial marker unit in a threaded fastening manner.

11. The apparatus of claim 8, wherein the holder unit is rotationally engaged with the fiducial marker unit in a bayonet coupling.

12. A method for mounting a fiducial marker for an image guided procedure using the apparatus for mounting a fiducial marker for an image-guided procedure of any one of claims 1 to 7, the method comprising:
engaging the holder unit to the fiducial marker unit connected to one side of the guide unit through the guide unit;
fixing the fiducial marker unit to an in-body target through the holder unit;
separating the holder unit from the fiducial marker unit; and
removing the holder unit from the body so that a portion of the guide unit is exposed outside the body.

13. The method of claim 12, further comprising:
inserting the guide unit exposed outside the body into the holder unit;
re-engaging the holder unit to the fiducial marker unit along the inserted guide unit; and
retrieving the fiducial marker unit from the body.
